(19) United States
(12) Patent Application Publication
(10) Pub. No.: US 2016/0346124 A1
(43) Pub. Date: Dec. 1, 2016

(54) Method of Generating a Pulse Sequence with a Pulse Control Apparatus for an Ophthalmic Surgical System

(71) Applicant: Carl Zeiss Meditec AG, Jena (DE)

(72) Inventors: Christoph Kuebler, Oberkochen (DE); Michael Eichler, Aalen (DE)

(21) Appl. No.: 15/231,348

(22) Filed: Aug. 8, 2016

Related U.S. Application Data
(63) Continuation of application No. 14/229,581, filed on Mar. 28, 2014.
Application No. 14/229,581 is a continuation of application No. PCT/EP2012/004054, filed on Sep. 27, 2012.

(30) Foreign Application Priority Data
Sep. 29, 2011 (DE) 10 2011 114 524.2

(51) Int. Cl. A61F 9/007

(52) U.S. Cl. CPC A61F 9/00745; A61B 2017/00181

(57) ABSTRACT

An ophthalmic surgical pulse control apparatus has a pulse generator, which, during a switch-on duration of the pulse generator, generates pulses having a pulse duration during which a needle of a phacoemulsification handpiece substantially vibrates at resonance and which, during a switch-on duration of the pulse generator, produces pulse pauses having a pulse pause duration during which the needle vibrates only minimally or not at all. A pulse with a follow-on pulse pause forms a pulse packet having a pulse packet duration. The pulse generator generates pulse packets which immediately follow one another and the sequence of the values of pulse duration and pulse pause duration of sequential pulse packets during the entire switch-on duration of the pulse generator is an aperiodic sequence.

### CROSS REFERENCE TO RELATED APPLICATIONS

This application is a continuation application of U.S. patent application Ser. No. 14/229,581, filed Mar. 28, 2014, which, in turn, is a continuation application of international patent application PCT/EP2012/004054, filed Sep. 27, 2012, designating the United States and claiming priority from German application 10 2011 114 524.2, filed Sep. 29, 2011, and the entire content of the above applications is incorporated herein by reference.

### FIELD OF THE INVENTION

The invention relates to an ophthalmic surgical pulse control apparatus and an ophthalmic surgical system including such a pulse control apparatus.

### BACKGROUND OF THE INVENTION

There are a number of surgical techniques for treating clouding within the eye lens, which is referred to as a cataract in medicine. The most common technique is phacoemulsification, in which a thin needle is introduced into the diseased lens and excited to vibrate by means of ultrasound. The vibrating needle emulsifies the lens in its direct vicinity in such a way that the created lens particles can be suctioned away through a line via a pump. Once the lens has been completely emulsified, a new artificial lens can be inserted into the empty capsular bag, and so a patient treated thus can regain good visual acuity.

In practice, comminuting a diseased lens by a needle vibrating with ultrasound works quite well. The higher the amount of energy supplied to the needle and the longer the ultrasonic vibration lasts, the faster small particles, which can be subsequently suctioned away, can be produced from a lens. However, a disadvantage here is that a relatively high temperature is generated in the surroundings of the vibrating needle in the case of such a high energy influx. Since the needle has to pierce through the cornea for surgery, this can lead to a corneal burn, which needs to be avoided at all costs. Furthermore, small lens particles can be pushed away from the needle tip in the case of a high energy influx with a large amplitude of the needle vibration. Therefore, the vibrating energy of the needle is converted into movement energy of small particles rather than comminuting and suctioning these away. This likewise leads to an increase of the temperature in the eye. Although such a temperature increase can be avoided by virtue of operating at a relatively low ultrasonic energy, this significantly increases the surgery duration. Moreover, it is not possible to comminute relatively large and hard particles.

### SUMMARY OF THE INVENTION

It is an object of the invention to provide an ophthalmic surgical pulse control apparatus, via which a needle of a phacoemulsification handpiece can be actuated in such a way that as many particles as possible can be produced and suctioned away in the case of a low energy influx and a short surgery duration.

The object is achieved by an ophthalmic surgical pulse control apparatus according to the invention which includes a pulse generator, which, during a switch-on duration of the pulse generator, is configured to produce pulses with a pulse duration during which the needle of a phacoemulsification handpiece substantially vibrates in resonance, and which, during the switch-on duration of the pulse generator, is configured to produce pulse pauses with a pulse pause duration during which the needle vibrates only minimally or not at all, wherein a pulse with a subsequent pulse pause forms a pulse packet with a pulse packet duration, wherein the pulse generator is configured to produce pulse packets which immediately follow one another, wherein the sequence of the values of pulse duration and pulse pause duration of successive pulse packets during the entire switch-on duration of the pulse generator forms an aperiodic sequence.

Such an aperiodicity of a sequence of the values of pulse duration and pulse pause duration leads to there being no repetition of a pulse pattern during the entire switch-on duration of the pulse generator. There is no period duration or frequency of a pulse pattern. This renders it possible that both small and large lens particles with both a high and a low degree of hardness can be comminuted and suctioned away well. Therefore, if a lens has a hard zone only in part, it is not necessary, for good measure, to carry out the operation at the high energy with a repeating sequence of the values of pulse duration with, in each case, a subsequent pulse pause duration. As a result of the aperiodic sequence of the values of pulse duration and pulse pause duration of successive pulse packets, there are a sufficient number of pulses during the entire switch-on duration of the pulse generator which, for example, only have a short duration and only introduce a small amount of energy into the eye such that soft lens parts can also be comminuted and emulsified. Furthermore, this also includes pulses which have a longer duration and are connected with a higher energy influx, and so it is also possible to comminute large and hard particles. Furthermore, the aperiodicity prevents standing waves from being formed relative to the tip of the needle, and so no additional local heating is produced. Furthermore, there are a sufficient number of pulse packets during an aperiodic sequence of the values of pulse duration and pulse pause duration, in the case of which small particles are not pushed away from the tip of the phacoemulsification needle but rather can be suctioned away well. Therefore, the pulse control apparatus according to the invention renders it possible to produce and suction away many particles with different characteristics while having a low energy influx and a short surgery duration.

Preferably, the values of pulse duration and pulse pause duration of a pulse packet are different from the values of pulse duration and pulse pause duration of an immediately following pulse packet. This ensures that there never is a succession of two pulse packets with the same pulse times, and so there is not a short-term repetition of pulse packets either.

In accordance with a further embodiment, a pulse duration and a pulse pause duration respectively only occur a single time during the aperiodic sequence of the values of pulse duration and pulse pause duration. This can achieve a sequence of the values of pulse durations or a sequence of the values of pulse pause durations with a linearly increasing, a linearly decreasing, a logarithmic or an exponential profile. This enables a linearly increasing, linearly decreasing, logarithmic or exponential energy influx, which can be advantageous for emulsifying lenses with very different hardness regions. By way of example, in the case of an exponentially increasing profile of the pulse durations and an exponentially increasing profile of the pulse pause durations, very intensive comminuting of lens particles can be carried out at the beginning with short pulse packets, wherein the pulse durations and pulse pause durations increase with increasing time duration. Therefore, the time provided for cooling down also lengthens with increasing time duration.

Preferably, the values of the pulse duration of successive pulse packets vary around a predetermined mean value with a predetermined positive and negative deviation therefrom. If a surgeon, on account of a preliminary examination, knows that, in the case of an, for example, older patient, there is a lens with a relatively high hardness, the surgeon can set the mean value of an average pulse duration in such a way that it is likely that enough energy is available for emulsifying the hard lens. However, if a preliminary examination yields that the patient has a very soft lens, the mean value of the pulse duration of successive pulse packets can be set to a low value such that relatively little energy is still supplied. This avoids unnecessarily large quantities of energy and therefore heat being introduced into the eye.

Preferably, the ratio of the values of pulse duration to pulse pause duration can be set to a predetermined value, which is greater than 0.01. This restricts the sequence of the values of pulse duration and pulse pause duration in such a manner that a minimum value of energy is always supplied.

The pulse control apparatus can also be embodied in such a way that the number of pulses per unit time can be set. In the case of a hard lens, it is possible to use a relatively large number of pulses per unit time, whereas work can be carried out with a small number of pulses per unit time in the case of a soft lens.

In the pulse control apparatus, energy supplied to the phacoemulsification handpiece during the pulse duration of a pulse packet can differ from energy supplied during the pulse duration of an immediately following pulse packet. This once again increases the variability of the apparatus. If there is a lens with very hard regions, but also very soft regions, this can achieve a particularly short surgery duration.

The object is also achieved by an ophthalmic surgical system, which includes a pulse control apparatus as described above and moreover includes a fluid control device, a power supply, a phacoemulsification handpiece, an input unit and a central control unit, which is coupled to the pulse control apparatus, the fluid control device, the power supply, the phacoemulsification handpiece and the input unit.

### BRIEF DESCRIPTION OF THE DRAWINGS

The invention will now be described with reference to the drawings wherein:

FIG. 1 shows a sequence of pulse duration and pulse pause duration of successive pulse packets;

FIG. 2 is a diagram which shows the respective duration of the pulses and pulse pauses for the pulse packets in accordance with FIG. 1;

FIG. 3 is a diagram which shows the pulse duration of successive pulse packets as a function of time;

FIG. 4 is a diagram which shows the supplied energy of successive pulse packets as a function of time;

FIG. 5 shows a further sequence of pulse duration and pulse pause duration of successive pulse packets;

FIG. 6 is a diagram which shows the respective duration of the pulses and pulse pauses for the pulse packets in accordance with FIG. 5;

FIG. 7 shows a further sequence of pulse duration and pulse pause duration of successive pulse packets;

FIG. 8 is a diagram which shows the respective duration of the pulses and pulse pauses for the pulse packets in accordance with FIG. 7; and,

FIG. 9 is a schematic view of an ophthalmic surgical system according to the invention.

### DESCRIPTION OF THE PREFERRED EMBODIMENTS OF THE INVENTION

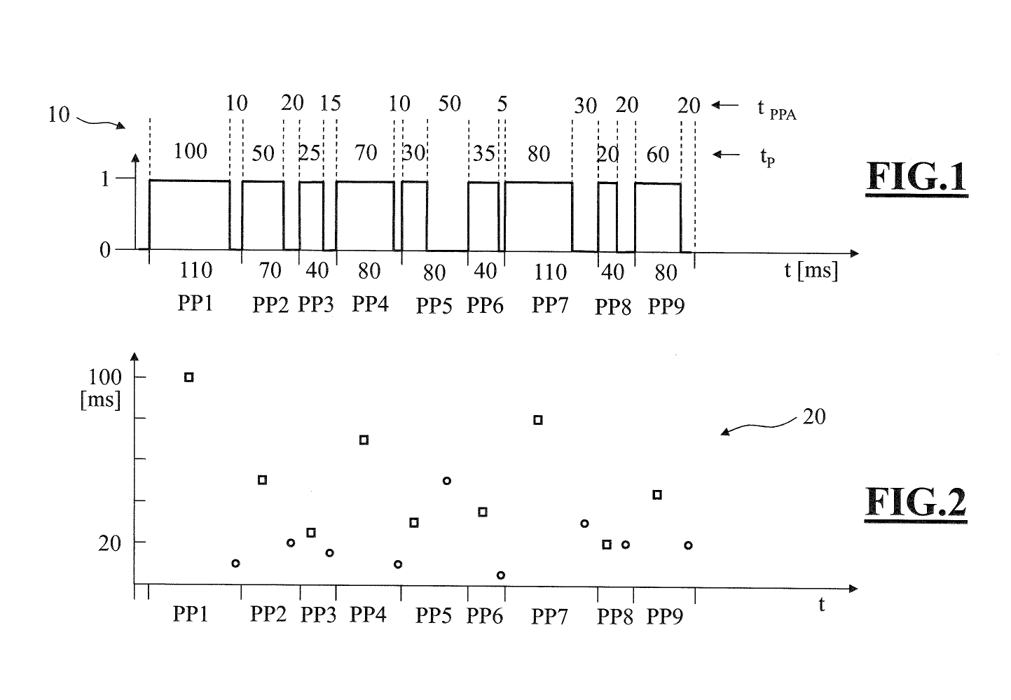

FIG. 1 depicts a diagram 10 which shows a sequence of pulse duration and pulse pause duration of successive pulse packets PP. The ordinate indicates the “no pulse” state using “0” and the “pulses switched on” state using “1”. The first pulse packet PP1 has a pulse packet duration of, for example, 110 milliseconds (ms), wherein the pulse duration is 100 ms and the pulse pause duration is 10 ms. This is immediately followed by a second pulse packet PP2 with a pulse packet duration of 70 ms, wherein the pulse duration is 50 ms and the pulse pause duration is 20 ms. Subsequently, this is followed by a third pulse packet PP3 with a pulse packet duration of 40 ms, wherein the pulse duration is 25 ms and the pulse pause duration is 15 ms. FIG. 1 shows further following pulse packets PP4 to PP9 with the respective pulse packet durations and the respective pulse duration and pulse pause duration. Therefore, this results in a sequence of the values of pulse duration and pulse pause duration as follows (numerical data in milliseconds):
100, 10, 50, 20, 25, 15, 70, 10, 30, 50, 35, 5, 80, 30, 20, 20, 60, 20.

It is easy to see that this sequence of the values of pulse duration and pulse pause duration of successive pulse packets is an aperiodic sequence. There is no regular pattern; vibration with a period does not exist.

In the above-described sequence there therefore are a total of 9 pulse packets during the whole switch-on duration of the pulse generator. The pulse generator is subsequently switched off. After a renewed switch on, the pulse control apparatus according to the invention causes an aperiodic sequence of the values of pulse duration and pulse pause duration to be present again during the whole switch-on duration of the pulse generator.

FIG. 2 shows a diagram 20, in which the duration of the pulses and the pulse pauses is plotted on the ordinate axis and the respective pulse packets specified in FIG. 1 are plotted on the abscissa axis. The respective pulse duration is represented by a square symbol while the respective pulse pause duration is represented by a circular symbol. The diagram clearly shows that there is an aperiodic sequence of the values of pulse duration and pulse pause duration during the entire switch-on duration of the pulse generator.

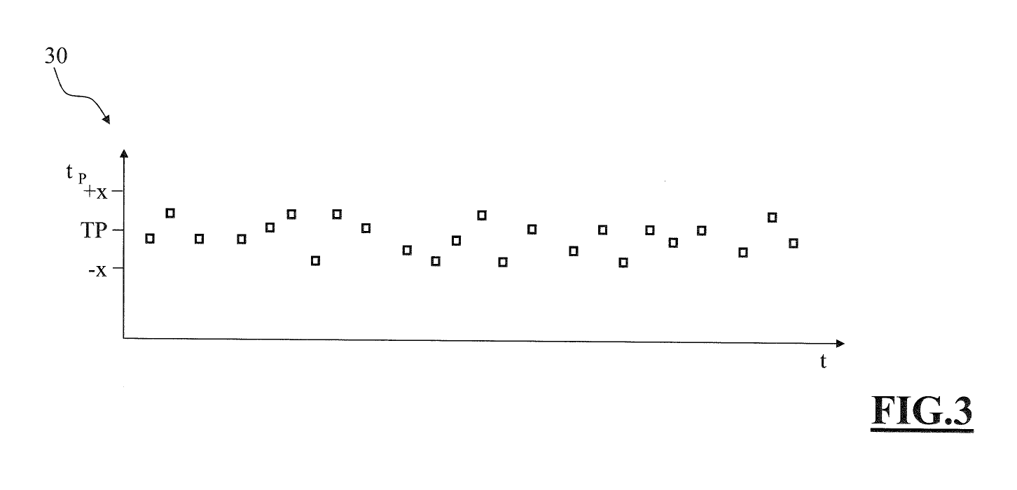

FIG. 3 depicts a diagram 30 which shows the values of the pulse duration of successive pulse packets. The values of the pulse duration of successive pulse packets in this case varies around a predetermined mean value TP with a predetermined positive deviation +x and negative deviation −x. This aperiodic sequence shows that the pulse duration never sinks to very low values. By way of example, this is expedient if a relatively hard lens is to be emulsified, and so the ophthalmologist already knows before the start of surgery that work should only be carried out with a relatively long pulse duration.

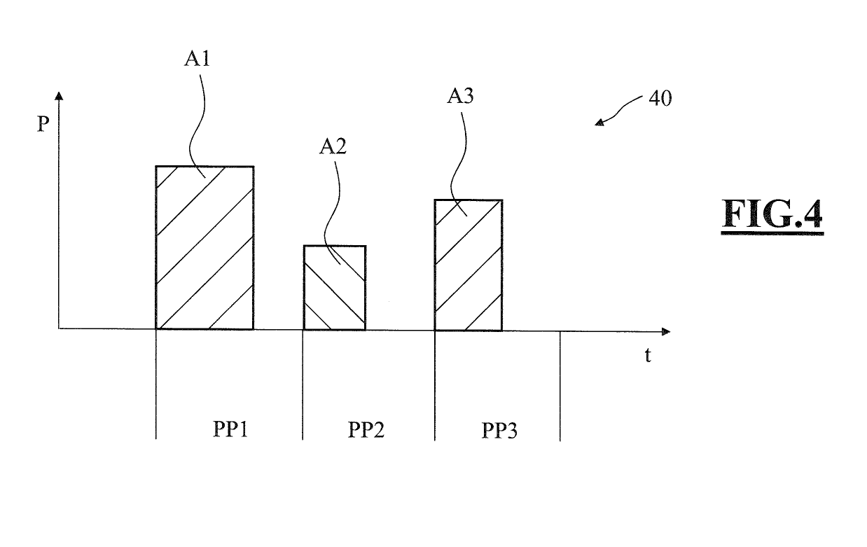

FIG. 4 depicts a diagram 40 which depicts the supplied energy P of successive pulse packets PP1 to PP3 as a function of time. The area A1 means a relatively high energy, whereas the subsequent area A2 during a subsequent pulse duration indicates a relatively low energy. The third pulse packet exhibits a pulse duration with a supplied energy in accordance with the area shaded with A3, which differs from the area A1 and A2. The variation in the aperiodic sequence of the values of individual pulse durations and pulse pause durations can additionally be increased by the differently supplied energy.

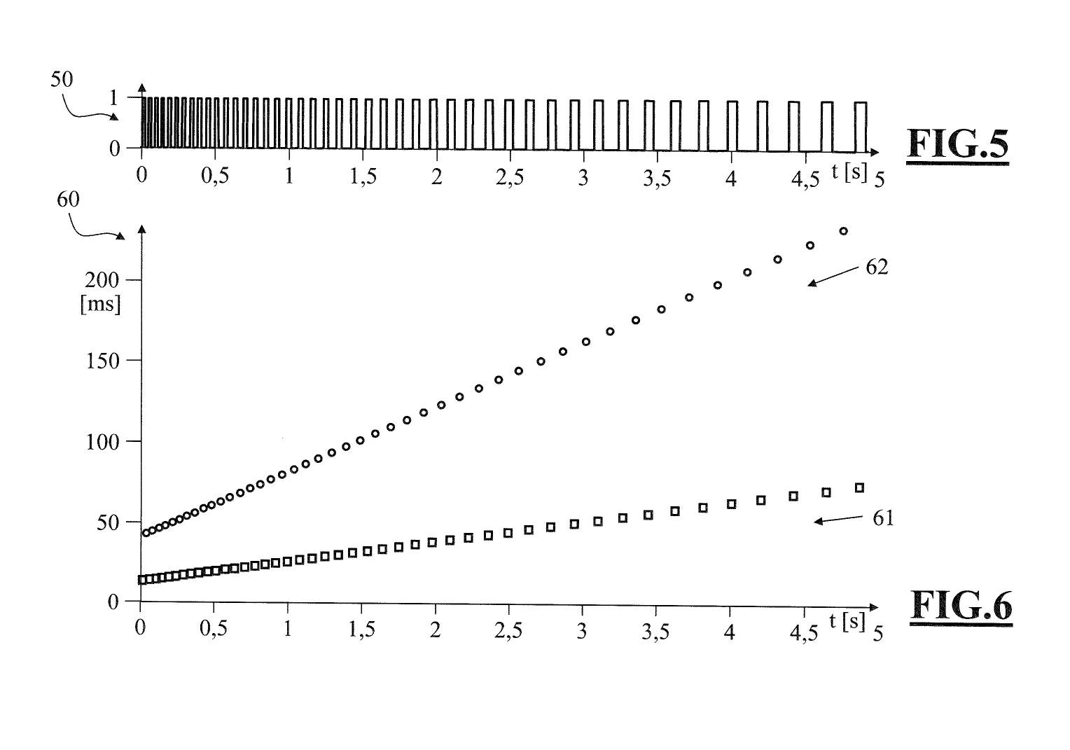

FIG. 5 depicts a diagram 50, which shows a sequence of pulse duration and pulse pause duration of successive pulse packets. The entire switch-on duration of the pulse generator is 5 seconds in this example. From the associated diagram 60 in FIG. 6, it is possible to identify that the sequence of the values of pulse durations—see reference sign 61—and the sequence of the values of the pulse pause durations—see reference sign 62—of the respective pulse packets increase linearly. The value of a pulse duration of a pulse packet therefore differs from the value of the subsequent pulse duration of a subsequent pulse packet. The same applies analogously to the pulse pause durations following one another. The ratio of a value of a pulse duration to the value of a subsequent pulse pause duration of a pulse packet differs from the ratio of a value of a pulse duration to the value of a subsequent pulse pause duration of an immediately following pulse packet.

In the example depicted in FIG. 6, the pulse pause duration increases with increasing number of pulse packets; this is sensible from a medical point of view. Using this, the cooling down time becomes ever longer with increasing surgery duration in the case of a needle of a phaco handpiece to which pulse packets are applied without interruption. By way of example, this avoids burns to the cornea due to the vibrating needle.

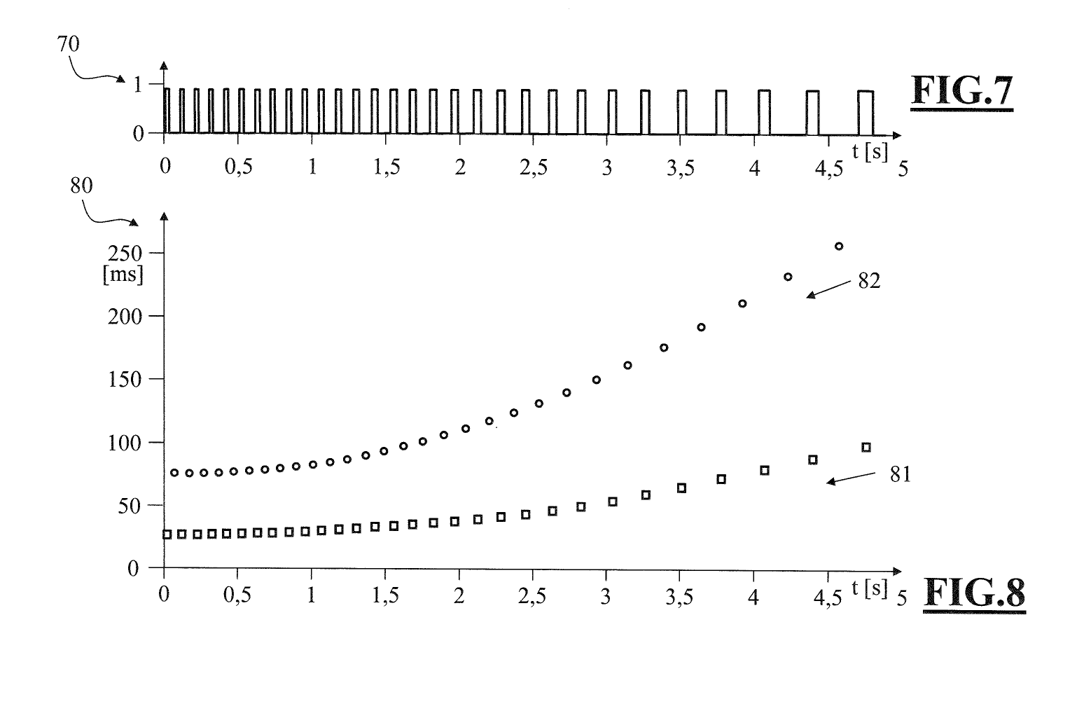

A further example is depicted in FIG. 7 using a diagram 70, which shows a sequence of the values of pulse duration and pulse pause duration of successive pulse packets. In FIG. 8, the associated time durations are plotted for the pulses and pulse pauses in accordance with FIG. 7. From FIG. 8, it is possible to identify that the sequence of the values of pulse durations—see reference sign 81—and the sequence of the values of the pulse pause durations—see reference sign 82—of the respective pulse packets increase exponentially. Here, a value of a pulse duration of a pulse packet also differs from the value of a subsequent pulse duration of a subsequent pulse packet. This likewise applies to the pulse pause durations following one another.

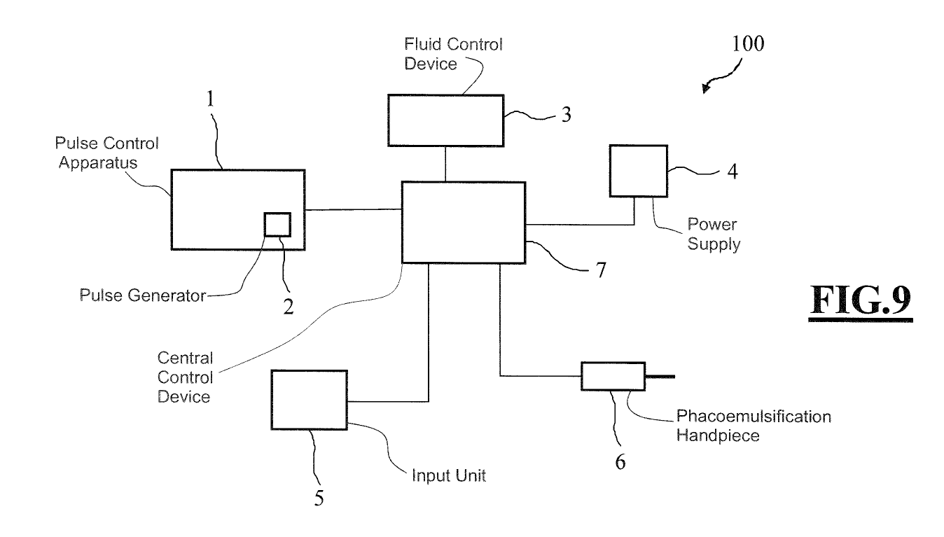

FIG. 9 depicts a schematic illustration of an ophthalmic surgical system 100, which includes a pulse control apparatus 1 with a pulse generator 2, a fluid control device 3, a power supply 4, an input unit 5, a phacoemulsification handpiece 6 and a central control device 7, which connects the aforementioned components to one another.

It is understood that the foregoing description is that of the preferred embodiments of the invention and that various changes and modifications may be made thereto without departing from the spirit and scope of the invention as defined in the appended claims.

1-9. (canceled)

10. A method of generating a pulse sequence comprising:
providing a pulse control apparatus for an ophthalmic surgical system;
generating a sequence of pulse packets with said pulse control apparatus; and,
actuating a needle of a phacoemulsification handpiece with said sequence of pulse packets;
wherein each pulse packet includes a pulse having a pulse duration and a pulse pause having a pulse pause duration;
wherein said sequence of pulse packets is aperiodic; and
wherein the pulse durations within said sequence of pulse packets vary around a predetermined mean value TP with a deviation x such that TP−x≦pulse duration ≦TP+x.

11. The method of generating a pulse sequence according to claim 10, further comprising:
generating said sequence of pulse packets during a switch-on duration of said pulse control apparatus.

12. The method of generating a pulse sequence according to claim 10, wherein said pulse packets within said sequence of pulse packets immediately follow one another.

13. The method of generating a pulse sequence according to claim 10, wherein the aperiodicity of said sequence of pulse packets prevents a formation of standing waves relative to a tip of said needle.

14. The method of generating a pulse sequence according to claim 10, wherein said pulse duration and said pulse pause duration are different for two pulse packets immediately following each other.

15. The method of generating a pulse sequence according to claim 10, wherein a value of a pulse duration and a value of a pulse pause duration occur a single time during said sequence of pulse packets.

16. The method of generating a pulse sequence according to claim 15, wherein the pulse durations and the pulse pause durations within said sequence of pulse packets increase linearly, logarithmically, or exponentially.

17. The method of generating a pulse sequence according to claim 15, wherein the pulse durations and the pulse pause durations within said sequence of pulse packets decrease linearly, logarithmically, or exponentially.

18. The method of generating a pulse sequence according to claim 10, wherein a ratio of pulse duration and pulse pause duration for each pulse packet is greater than 0.01.

19. The method of generating a pulse sequence according to claim 10, further comprising:
setting a predetermined number of pulses per unit time.

20. The method of generating a pulse sequence according to claim 10, further comprising:
varying an energy supplied during a pulse duration between two pulse packets immediately following each other.
